(19) United States
(12) Patent Application Publication
(10) Pub. No.: US 2008/0051896 A1
(43) Pub. Date: Feb. 28, 2008

(54) Expandable Spinous Process Distractor

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/832,989

(22) Filed: Aug. 2, 2007

Related U.S. Application Data
(60) Provisional application No. 60/823,595, filed on Aug. 25, 2006.

(51) Int. Cl. A61F 2/44

(52) U.S. Cl. 623/17.12; 623/17.16

(57) ABSTRACT

An expandable device is positioned surgically between spinous processes, permitting the incremental distraction of posterior spinal elements when the device is filled with a hardenable material.

This application claims benefit of U.S. Provisional Application No. 60/823,595, filed on Aug. 25, 2006.

This invention relates to a expandable spinous process distractor.

### BACKGROUND OF THE INVENTION

This invention relates to the field of orthopedic spine surgery and particularly to the technique of spinous process distraction, serving to unload the posterior annulus, distract facets and open neural foramina as well as enlarging the cross-sectional area of the central spinal canal.

Of all animals processing a backbone, human beings are the only creatures who remain upright for significant periods of time. From an evolutionary standpoint, this erect posture has conferred a number of strategic benefits, not the least of which is freeing the upper limbs for purposes other than locomotion. From and anthropologic standpoint, it is also evident that this unique evolutionary adaptation is a relatively recent change and as such has not benefited from natural selection as much as have backbones held in the horizontal attitude. As a result, the stresses acting upon the human backbone (or “vertebral column”), are unique in many senses, and result in a variety of problems or disease states that are peculiar to the human species.

The human vertebral column is essentially a tower of bones held upright by fibrous bands called ligaments and contractile elements called muscles. There are seven bones in the neck or cervical region, twelve in the chest or thoracic region, and five in the low back or lumbar region. There are also five bones in the pelvis or sacral region which are normally fused together and form the back part of the pelvis. This column of bones is critical for protecting the delicate spinal cord and nerves, and for providing structural support for the entire body.

Between the vertebral bones themselves exist soft tissue structures—discs—composed of fibrous tissues and cartilage which are compressible and at as shock absorbers for sudden downward forces on the upright column. More importantly, the discs allow the bones to move independently of each other to permit functional mobility of the column of spinal vertebrae. Unfortunately, the repetitive forces which act on these intervertebral discs during repetitive day-to-day activities of bending, lifting and twisting cause them to break down or degenerate over time.

Presumably because of the human upright posture, the intervertebral discs have a high propensity to degenerate. Overt trauma, or covert trauma occurring in the course of repetitive activities disproportionately affect the more highly mobile areas of the spine. Disruption of a disc's internal architecture leads to bulging, herniation or protrusion of pieces of the disc and eventual disc space collapse. Resulting mechanical and even chemical irritation of surrounding neural elements (spinal cord and nerves) cause pain, attended by varying degrees of disability. In addition, loss of disc space height relaxes tension on the longitudinal spinal ligaments thereby contributing to varying degrees of spinal instability. This ligamentous laxity and loss of disc space height in turn causes a loss of the cross-sectional area of the neural foramina and a pathologic increase in the forces acting on the spinal facet joints. As a consequence, the ligaments undergo a compensatory hypertrophy, which, coupled with the degenerative hypertrophy of the facet joints and bulging of the degenerative intervertebral discs, leads to a net decrease in the cross-sectional area of the central spinal canal. As a further consequence of this degenerative narrowing of the central spinal canal and neural foramina, various neurologic syndromes arise, not the least of which include sciatica and neurogenic claudication.

The time honored surgical treatment of this spectrum of degenerative changes has largely focused on the surgical opening or enlargement of the central canal (laminectomy) and neural spinal foramina (foraminotomy). These operations are considered major surgeries attended by significant risk. Because the sufferers of these conditions are generally elderly, lesser procedures have been sought to treat these conditions thereby shortening recovery and lowering the overall risk.

Recently, it has been noted that distraction of the spinous processes serves to sufficiently unload posterior elements to achieve neural foramina enlargement and improvement in the cross-sectional area of a narrowed central spinal canal.

Present techniques of spinous process distraction rely on the open surgical placement of a space occupying object between adjacent spinous processes to achieve sufficient separation between them to achieve foramina and central spinal enlargement. This technique necessitates that the object have a sufficient diameter to achieve necessary spinal distraction to achieve these goals. As a result, the device must be implanted through an open surgical incision and various sized implants must be necessary to achieve appropriate distraction in all cases because of individual variability.

### SUMMARY OF THE INVENTION

It is an object of this invention to provide for an interspinous distraction apparatus that can be implanted through either open or percutaneous techniques. It is also an object of this invention to have an implant that can address the infinite variation between human spinous processes such that multiple sized implants can be avoided.

By having the device distract via an expandable mechanism, the infinite variety between individual spinous processes can be addressed with a single implant thereby minimizing the need for multiple sized implants. Additionally, by allowing the implant to be placed in a contracted state and then enlarged in situ, a percutaneous technique may be employed thus obviating the need for a surgical incision and open surgical dissection.

To achieve these objectives a device is implanted via a mini-open or percutaneous techniques between adjacent spinous processes. Direct vision is used in open techniques and radiographic or fluoroscopic views are utilized when percutaneous techniques are preformed.

The device itself is designed to expand in a hydraulic fashion when filled with a liquid or semi-liquid material which forces the intrinsic elements apart.

In one embodiment, the expansile element is a distensible sac having either elastomeric or no elastomeric properties. The sac is initially filled with a radiopaque liquid to judge the volume of material necessary to achieve optimal spinous process distraction. Once the exact volume is known, the radiopaque liquid is aspirated and replaced with a hardenable material that hardens to a stiff state if rigid fixation is desired or to a gel state if some controlled movement of adjacent spinal elements is to be permitted. In either situation, the net result is distraction of adjacent spinous processes through a spectrum of degrees to achieve the enlargement of the central spinal canal and associated neural foramina.

In the second embodiment shown in FIGS. 6-8, two opposite facing yokes are distracted by a piston/cylinder mechanism which in turn is expanded through hydraulic means. The yokes fit around the spinous processes and are then incrementally separated as the piston is displaced by the filling of the cylinder. The cylinder, in turn, expands in a telescoping fashion so that differing degrees of separation or distraction can be achieved. As in the first embodiment, the initial filling of the cylinder is done with a radiopaque liquid and the optimal distraction of spinous elements relative to the volume filling the cylinder is noted. Once the volume is known, the radiopaque liquid is aspirated and then the cylinder is filled with a similar volume of hardenable material, said hardenable material allowing rigid fixation if the final state is to be stiff or relative fixation if the hardenable material sets to a gel like state.

### BRIEF DESCRIPTION OF THE DRAWINGS

In the accompanying drawings,

FIG. 1 is a side elevation of an expandable spinous process distractor embodying the invention, showing a cannula having an inner removable stylet being positioned between two spinous processes;

FIG. 2 demonstrates the stylet having been withdrawn and replaced with a deflated sac or balloon;

FIG. 3 demonstrates the cannula being withdrawn and the balloon left in situ between the spinous processes;

FIG. 4 demonstrates the balloon being inflated and slowly separating the spinous processes;

FIG. 5 demonstrates the final position of the balloon after optimal distraction of the spinous processes has been achieved;

FIG. 6 demonstrates a second embodiment of the invention, in its collapsed state positioned between adjacent spinous processes;

FIG. 7 demonstrates the device in a semi expanded state; and

FIG. 8 demonstrates the device in its fully expanded state.

### DESCRIPTION OF THE PREFERRED EMBODIMENT

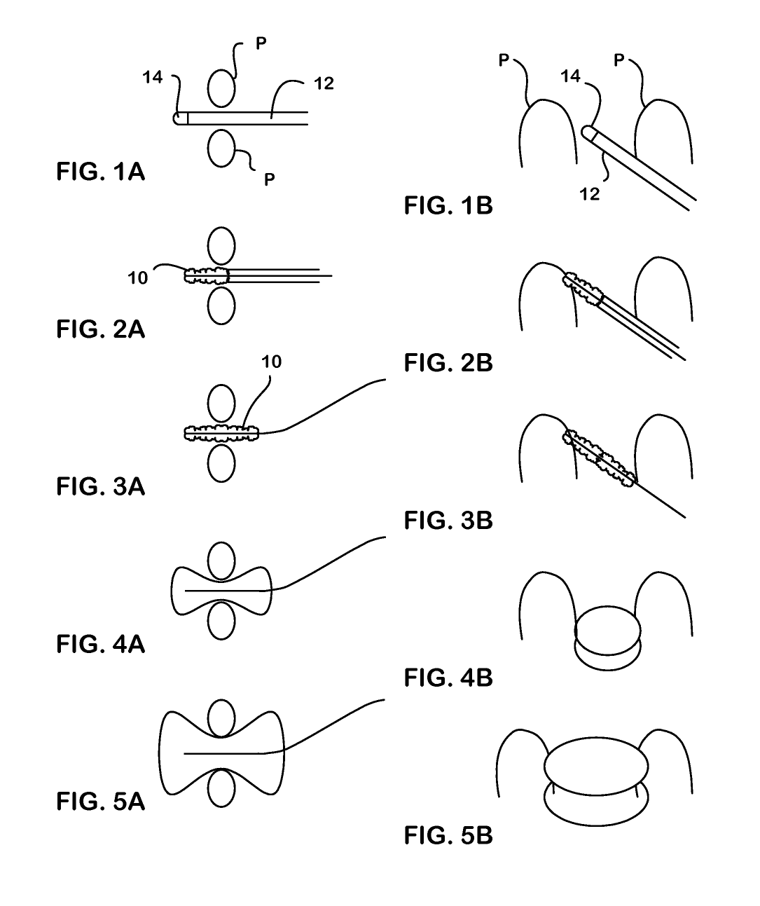

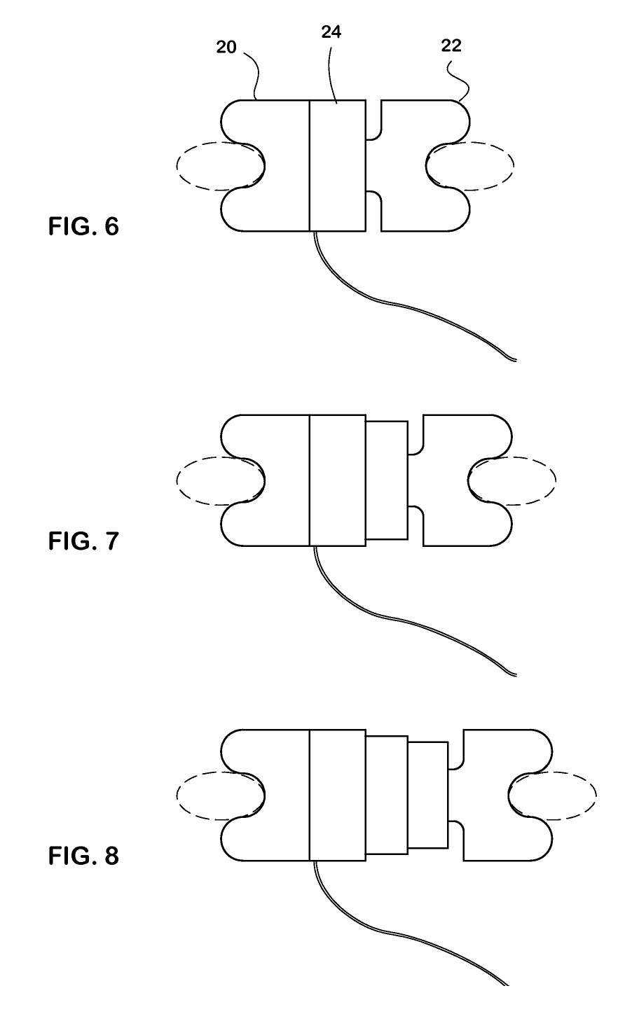

A expandable spinous process distractor embodying the invention is shown in FIGS. 1-5.

In this first embodiment a distensible sac or balloon 10 is inserted between the spinous processes “P” via an open or percutaneous technique. In the open technique, this is accomplished under direct vision, whereas in the percutaneous technique, this is achieved using x-ray fluoroscopy.

To position the sac or balloon, a cannula 12 with a stylet 14 is initially placed between the spinous processes. Once in position, the stylet is removed from the cannula and the deflated balloon 10 is slid into position along the cannula, which is then withdrawn, leaving the balloon positioned between adjacent spinous processes. The balloon is then filled with a radiopaque fluid (not shown), fed to the balloon through an inflation tube 16, so that it distends the fundus of the balloon which then expands between the spinous processes. As the balloon is filled further, the spinous processes are slowly separated from each other. Because the balloon displaces surrounding soft tissues easier than bone, the pliable wall of the balloon assumes a dumbbell shape that subsequently fixes it in position between the spinous processes and prevents dislodgement. Once optimal distraction has been achieved—as determined by direct vision in the open technique or by x-ray in the percutaneous technique—the volume of fluid is noted and recorded.

The fluid is then replaced with an equal volume of hardenable material (not shown) and allowed to set, keeping the spinous processes in a permanently distracted state. The tube 12 used to insufflate the balloon is then detached leaving it in situ between the spinous processes. The balloon is now filled with a hardenable material and the insufflating tube has been detached and removed. The balloon's dumbbell shape keeps it securely fixated in position.

In the second embodiment shown in FIGS. 6-8, two yokes 20, 22 are connected to one another by a hydraulic cylinder 24 that expands in a telescoping fashion when fluid is injected into the telescoping component. As the telescoping component expands, the yokes are forced away from each other in a graduated fashion.

When the yokes 20, 22 are positioned against adjacent spinous processes P and the telescoping component 24 is filled with fluid, gradual and optimal distraction of the spinous processes can be achieved. Once optimal distraction is achieved by direct vision or via fluoroscopic x-ray, the fluid can be withdrawn and then replaced with a hardenable material that sets and fixates the device in position between the spinous processes. The yokes prevent dislodgement of the device so that, once the injected material hardens, permanent distraction of the spinous processes is achieved.

Since the invention is subject to modifications and variations, it is intended that the foregoing description and the accompanying drawings shall be interpreted as only illustrative of the invention defined by the following claims.

I claim:

1. An expandable spinous process distractor comprising
a device comprising an expandable chamber to allow adjustable distraction of spinous processes via open or percutaneous techniques.

2. The invention of claim 1, wherein the expansible chamber is a distensible sac or balloon.

3. The invention of claim 1, wherein the device is a telescopic cylinder expandable with hydraulic means.

4. The invention of claim 1, wherein the expansible chamber is electrometric and expands along paths of least resistance.

5. The invention of claim 1, wherein the expansible chamber is non-electrometric and expands in a predetermined fashion designed to achieve optimal distraction of spinous processes by virtue of its final shape.

6. The invention of claim 1, further comprising a hardenable material which, when inserted into the balloon, renders it stiff and incompressible.

7. The invention of claim 1, wherein the balloon is constructed so as to form a dumbbell shape that fixes it in position between the spinous processes and prevent dislodgement.

8. The invention of claim 6, wherein the hardenable material is compressible or pliable, so as to permit some controlled motion between the processes.
